(19) United States
(12) Patent Application Publication
(10) Pub. No.: US 2023/0201281 A1
(43) Pub. Date: Jun. 29, 2023

(54) VAGINAL MICROBIOTA COMPOSITIONS

(71) Applicant: FERRING B.V., Hoofddorp (NL)

(72) Inventors: Dana M. WALSH, Maplewood, MN (US); Heidi HAU, St. Paul, MN (US); Kristi STRANDBERG, St. Paul, MN (US); Kenneth F. BLOUNT, Guildford, CT (US); Lee A. JONES, Fridley, MN (US)

(73) Assignee: FERRING B.V., Hoofddorp (NL)

(21) Appl. No.: 17/928,566

(22) PCT Filed: Jun. 1, 2021
(86) PCT No.: PCT/US2021/035245

§ 371 Date: Nov. 29, 2022

Related U.S. Application Data
(60) Provisional application No. 63/033,008, filed on Jun. 1, 2020.

(51) Int. Cl. A61K 35/747; A61K 9/00; A61K 9/02

(52) U.S. Cl. CPC A61K 35/747; A61K 9/0034; A61K 9/02

(57) ABSTRACT

Compositions and methods for treating patients are disclosed. An example composition may include a vaginal microbiota composition. The vaginal microbiota composition may include a mixture of bacteria. The mixture of bacteria may include Lactobacillus crispatus, Lactobacillus gasseri, Lactobacillus jensenii, and Lactobacillus rhamnosus.

### TECHNICAL FIELD

The present disclosure pertains to compositions and methods for treating patients.

### BACKGROUND

A wide variety of compositions and methods have been developed for treating diseases and/or conditions, for example diseases and/or conditions of the reproductive system, digestive track, liver, immune system, and the like. Of the known compositions and methods, each has certain advantages and disadvantages. There is an ongoing need to provide alternative compositions and methods for treating diseases and/or conditions.

### BRIEF SUMMARY

This disclosure provides design, material, manufacturing method, and use alternatives for compositions and methods for treating patients. A vaginal microbiota composition is disclosed. The vaginal microbiota composition comprises: a mixture of bacteria including Lactobacillus crispatus, Lactobacillus iners, Lactobacillus gasseri, Lactobacillus jensenii, Lactobacillus reuteri, Lactobacillus acidophilus, Lactobacillus vaginalis, Lactobacillus rhamnosus, Lactobacillus johnsonii, Lactobacillus helveticus, Lactobacillus plantarum, Lactobacillus fermentum, Lactobacillus salivarius, and Lactobacillus delbrueckii.

Alternatively or additionally to any of the embodiments above, the mixture of bacteria includes 25-75% Lactobacillus crispatus.

Alternatively or additionally to any of the embodiments above, the mixture of bacteria includes 30-60% Lactobacillus crispatus.

Alternatively or additionally to any of the embodiments above, the mixture of bacteria includes 40% Lactobacillus crispatus.

Alternatively or additionally to any of the embodiments above, the mixture of bacteria includes 10-40% Lactobacillus iners.

Alternatively or additionally to any of the embodiments above, the mixture of bacteria includes 15-30% Lactobacillus iners.

Alternatively or additionally to any of the embodiments above, the mixture of bacteria includes 20% Lactobacillus iners.

Alternatively or additionally to any of the embodiments above, the mixture of bacteria includes 1-10% Lactobacillus gasseri.

Alternatively or additionally to any of the embodiments above, the mixture of bacteria includes 2-8% Lactobacillus gasseri.

Alternatively or additionally to any of the embodiments above, the mixture of bacteria includes 5% Lactobacillus gasseri.

Alternatively or additionally to any of the embodiments above, the mixture of bacteria includes 1-10% Lactobacillus jensenii.

Alternatively or additionally to any of the embodiments above, the mixture of bacteria includes 2-8% Lactobacillus jensenii.

Alternatively or additionally to any of the embodiments above, the mixture of bacteria includes 5% Lactobacillus jensenii.

Alternatively or additionally to any of the embodiments above, the mixture of bacteria includes 0.1-5% Lactobacillus reuteri.

Alternatively or additionally to any of the embodiments above, the mixture of bacteria includes 0.5-4% Lactobacillus reuteri.

Alternatively or additionally to any of the embodiments above, the mixture of bacteria includes 1% Lactobacillus reuteri.

Alternatively or additionally to any of the embodiments above, the mixture of bacteria includes 0.1-5% Lactobacillus acidophilus.

Alternatively or additionally to any of the embodiments above, the mixture of bacteria includes or about 0.5-4% Lactobacillus acidophilus.

Alternatively or additionally to any of the embodiments above, the mixture of bacteria includes 1% Lactobacillus acidophilus.

Alternatively or additionally to any of the embodiments above, the mixture of bacteria includes 0.1-5% Lactobacillus vaginalis.

Alternatively or additionally to any of the embodiments above, the mixture of bacteria includes 0.5-4% Lactobacillus vaginalis.

Alternatively or additionally to any of the embodiments above, the mixture of bacteria includes 1% Lactobacillus vaginalis.

Alternatively or additionally to any of the embodiments above, the mixture of bacteria includes 0.1-5% Lactobacillus rhamnosus.

Alternatively or additionally to any of the embodiments above, the mixture of bacteria includes 0.5-4% Lactobacillus rhamnosus.

Alternatively or additionally to any of the embodiments above, the mixture of bacteria includes 1% Lactobacillus rhamnosus.

Alternatively or additionally to any of the embodiments above, the mixture of bacteria includes 0.1-5% Lactobacillus johnsonii.

Alternatively or additionally to any of the embodiments above, the mixture of bacteria includes 0.5-4% Lactobacillus johnsonii.

Alternatively or additionally to any of the embodiments above, the mixture of bacteria includes 1% Lactobacillus johnsonii.

Alternatively or additionally to any of the embodiments above, the mixture of bacteria includes 0.1-5% Lactobacillus helveticus.

Alternatively or additionally to any of the embodiments above, the mixture of bacteria includes 0.5-4% Lactobacillus helveticus.

Alternatively or additionally to any of the embodiments above, the mixture of bacteria includes 1% Lactobacillus helveticus.

Alternatively or additionally to any of the embodiments above, the mixture of bacteria includes 0.1-5% Lactobacillus plantarum.

Alternatively or additionally to any of the embodiments above, the mixture of bacteria includes 0.5-4% Lactobacillus plantarum.

Alternatively or additionally to any of the embodiments above, the mixture of bacteria includes or about 1% Lactobacillus plantarum.

Alternatively or additionally to any of the embodiments above, the mixture of bacteria includes 0.1-5% Lactobacillus fermentum.

Alternatively or additionally to any of the embodiments above, the mixture of bacteria includes 0.5-4% Lactobacillus fermentum.

Alternatively or additionally to any of the embodiments above, the mixture of bacteria includes 1% Lactobacillus fermentum.

Alternatively or additionally to any of the embodiments above, the mixture of bacteria includes 0.1-5% Lactobacillus salivarius.

Alternatively or additionally to any of the embodiments above, the mixture of bacteria includes 0.5-4% Lactobacillus salivarius.

Alternatively or additionally to any of the embodiments above, the mixture of bacteria includes 1% Lactobacillus salivarius.

Alternatively or additionally to any of the embodiments above, the mixture of bacteria includes 0.1-5% Lactobacillus delbrueckii.

Alternatively or additionally to any of the embodiments above, the mixture of bacteria includes 0.5-4% Lactobacillus delbrueckii.

Alternatively or additionally to any of the embodiments above, the mixture of bacteria includes 1% Lactobacillus delbrueckii.

Alternatively or additionally to any of the embodiments above, the mixture of bacteria includes one or more lyophilized strains of bacteria.

Alternatively or additionally to any of the embodiments above, the mixture of bacteria is disposed in a capsule.

Alternatively or additionally to any of the embodiments above, the mixture of bacteria is disposed in a suppository.

Alternatively or additionally to any of the embodiments above, the mixture of bacteria is disposed in a soluble shell.

A method for treating an infection is disclosed. The method comprises: administering the vaginal microbiota composition of any one of embodiments above to a patient with an infection.

Alternatively or additionally to any of the embodiments above, wherein the infection includes one or more of bacterial vaginosis, a candidiasis infection, a human papilloma virus infection, a urinary tract infection, a sexually transmitted infection, and a gynecological cancer.

A method for treating a patient is disclosed. The method comprises: administering the vaginal microbiota composition of any one of embodiments above to a patient.

Alternatively or additionally to any of the embodiments above, wherein administering the vaginal microbiota composition to the patient includes one or more of preventing preterm birth, preventing miscarriages, treating infertility, treating interstitial cystitis, and treating polycystic ovary syndrome.

A vaginal microbiota composition is disclosed. The vaginal microbiota composition comprises: a suppository; and a mixture of lyophilized bacteria disposed in the suppository, the mixture of lyophilized bacteria including Lactobacillus crispatus, Lactobacillus iners, Lactobacillus gasseri, Lactobacillus jensenii, Lactobacillus reuteri, Lactobacillus acidophilus, Lactobacillus vaginalis, Lactobacillus rhamnosus, Lactobacillus johnsonii, Lactobacillus helveticus, Lactobacillus plantarum, Lactobacillus fermentum, Lactobacillus salivarius, and Lactobacillus delbrueckii.

A vaginal microbiota composition bank is disclosed. The vaginal microbiota composition bank comprises: a first container including a first population of one or more microbes disposed therein, the first population being collected from a first donor; a second container including a second population of one or more microbes disposed therein, the second population being collected from a second donor; and an indexing system including donor data from the first donor and the second donor.

A vaginal microbiota composition bank is disclosed. The vaginal microbiota composition bank comprises: a first container including a first population of one or more microbes disposed therein, the first population being collected from a first donor; and a second container including a second population of one or more microbes disposed therein, the second population being collected from a second donor.

Alternatively or additionally to any of the embodiments above, the vaginal microbiota composition bank includes an indexing system including donor data from the first donor and the second donor.

A vaginal microbiota composition bank is disclosed. The vaginal microbiota composition bank comprises: a first container including a first population of one or more lyophilized microbes disposed therein, the first population being collected from a first donor; a second container including a second population of one or more lyophilized microbes disposed therein, the second population being collected from a second donor; and an indexing system including donor data from the first donor and the second donor.

A vaginal microbiota composition bank is disclosed. The vaginal microbiota composition bank comprises: a first container including a first population of one or more lyophilized microbes disposed therein, the first population being collected from a first donor; a second container including a second population of one or more lyophilized microbes disposed therein, the second population being collected from a second donor.

Alternatively or additionally to any of the embodiments above, the vaginal microbiota composition bank includes an indexing system including donor data from the first donor and the second donor.

The above summary of some embodiments is not intended to describe each disclosed embodiment or every implementation of the present disclosure. The Detailed Description, which follow, more particularly exemplify these embodiments.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The human microbiome (or human microbiota) is the aggregate of microorganisms that reside on the surface and in deep layers of skin, in the saliva and oral mucosa, in the conjunctiva, and in the gastrointestinal, genito-urinary, and/or vaginal tracts of humans inclusive of microorganisms related to reproductive health such as the placental microbiome. The human microbiome is comprised of bacteria but may also include fungi, phages, viruses, archaea, and the like. Some of these organisms perform tasks that are useful for the human host, but the function of the majority of the organisms that make up the human microbiome is still being studied. Under normal circumstances, these microorganisms do not cause disease to the human host, but instead participate in maintaining health.

The vaginal microbiota may play a role in a number of different health conditions. Some of these conditions may include infections such as bacterial vaginosis, candidiasis infection, human papilloma virus infections, urinary tract infections, and/or sexually transmitted infections. Other conditions may include gynecological cancers (e.g., cervical cancer), preterm birth, miscarriages, infertility, interstitial cystitis, polycystic ovary syndrome, and the like. Restoring, normalizing, and/or otherwise shifting the makeup of the vaginal microbiota may help to alleviate these and other conditions. Disclosed herein are compositions and methods for treating patients. At least some of the compositions include vaginal microbiota compositions. At least some of the methods include methods for treating bacterial vaginosis, treating candidiasis infections, treating human papilloma virus infections, treating urinary tract infections, treating sexually transmitted infections, treating gynecological cancers (e.g., cervical cancer), preventing preterm birth, preventing miscarriages, treating infertility, treating interstitial cystitis, treating polycystic ovary syndrome, and the like.

In healthy, reproductive-age women, bacteria of the genus Lactobacillus tend to dominate the vaginal microbiome. There are four species consistently identified across women; Lactobacillus crispatus, Lactobacillus iners, Lactobacillus gasseri, and Lactobacillus jensenii. These species are listed in order of their frequency of dominance among women of white, black, Asian, and Hispanic ethnicities. Generally, lactobacilli dominance of the vaginal microbiome is associated with health. This dominance of a single genus or species creates a low diversity microbiome. Increases in vaginal microbiome diversity are associated with poor health and a higher risk of infection. For example, bacteria from the genera Sneathia, Megasphaera, Atopobium, Peptoniphilus, Dialister, Prevotella, and/or Gardnerella may tend to correlate with an unhealthy vaginal microbiota, as they have been identified in women with other indicators of poor vaginal health (high Nugent score and vaginal pH). These generalizations, however, can differ in different populations.

The vaginal microbiome may vary between pre-puberty, pre-menopausal, and post-menopausal women. For example, pre-puberty women may have a relatively low relative abundance of Lactobacillus and a relatively high diversity in microbes (as well as have a relatively thin vaginal epithelium, a relatively thin layer of mucus, and a relatively low amount of glycogen). Pre-menopausal women may have a relatively high relative abundance of Lactobacillus and a relatively low diversity in microbes (as well as have a relatively thick vaginal epithelium, a relatively thick layer of mucus, relatively high estrogen levels, and a relatively low amount of glycogen). Post-menopausal women may have a moderate relative abundance of Lactobacillus and a relatively low or lower diversity in microbes (as well as have a relatively thin vaginal epithelium, a relatively thin layer of mucus, relatively moderate estrogen levels, and a relatively moderate amount of glycogen). Post-menopausal women symptomatic of a vaginal infection (e.g., bacterial vaginosis) may have a lower relative abundance of Lactobacillus and a relatively high diversity in microbes. Collectively, compositions that tend to include or otherwise increase the relative abundance of Lactobacillus, as well as help to lower the microbial diversity, may be beneficial.

In at least some instances, the compositions disclosed herein (e.g., vaginal microbiota compositions) utilize vaginal microbiota collected from vaginal microbiota donors. However, other sources of vaginal microbiota are contemplated including bacterial cultures, etc. Potential vaginal microbiota donors may be identified and may undergo a number of screening processes. In some instances, potential donors may be screened using a health history questionnaire. An example questionnaire may be the same as or similar to that used by the Red Cross for screening of potential blood donors. In some of these and in other instances, potential donors may be screened for common infectious diseases and other conditions. Such screening may include blood tests, stool tests, urine tests, vaginal swab tests and the like. Such tests may include testing for the presence of HIV, hepatitis (hepatitis A, B, and/or C), syphilis, C. difficile, bacterial pathogens, ova and parasites, and/or the like. These are just examples. The health of donors may be monitoring by administering blood tests, analyzing stool samples, analyzing urine samples, analyzing vaginal secretions, periodically updating health history, etc. Vaginal swabs may be collected to assess vaginal health via Gram staining and Nugent scoring, white blood cell counts, and detection of the presence of yeast. Urine and/or vaginal swabs may be collected to test for sexually transmitted infections.

After a potential donor has been determined to be healthy enough to donate, the donor is provided with a sample collection kit (e.g., a sample self-collection kit). The sample collection kit may include donor instructions, a vaginal DNA/RNA collection kit for sequencing analysis (e.g., OMNIgene vaginal collection kit ORM-130, commercially available from DNAgeneotek™), and a specimen collection and transportation kit (e.g., an eSwab 480C Copan Liquid Amies Elution Swab Collection and Transport System, commercially available from Copan Diagnostics). Donors may be asked to sign an informed consent form. Each donor may be asked to collect two samples. The first sample may be used for sequencing analysis. For example, after hand washing, a swab from the vaginal DNA/RNA collection kit can be used to collect the vaginal specimen by inserting the swab a few inches into the vagina and swabbing the vaginal wall for about 20 seconds. The swab can then be inserted into a tube containing a stabilizing buffer and the tube/specimen can be further analyzed. The second sample may be used for culturing vaginal microbes. For example, a swab from the specimen collection and transportation kit can used to collect the vaginal specimen. The swab can then be inserted into a tube containing liquid Amies solution (e.g., which may preserve aerobic, anaerobic, and fastidious bacteria for up to 48 hours). Upon collection, the donor places the sample tubes and materials inside a biohazard bag, and then drops the specimen off at the donation facility.

To process the sample for sequencing (e.g., shotgun sequencing), first the protease (e.g., QIAGEN Protease) may be rehydrated using sterile PCR-certified water to generate an 80 mg/mL solution (this may include inverting the sample 10 times or more). The sample (e.g., the vaginal DNA/RNA collection kit sample) may be removed from the refrigerator and vortexed for 30 seconds. The collection tube may be shook three times to bring the solution containing the sample to the bottom of the tube. Five microliters of the rehydrated protease may be added to the collection tube containing the vaginal swab sample. The sample may be inverted 10 or more times. The sample may then be incubated for 2 hours in a 50° C. incubator. The sample may then be vortexed for 30 seconds. The collection tube may then be shook three times to bring the solution containing the sample to the bottom of the tube. The swab may be removed from the tube (which may include pressing the swab on the side of the tube to recover sample absorbed into the swab). 500 microliters of the sample may be aliquoted into each of two 2-mL cryovials for storage and the samples can be stored at −80° C. until ready for sequencing analysis (which may be conducted at a suitable processing/analysis facility such as Divergent™ in St. Paul, Minn.).

To process the sample for culturing, the sample (e.g., the specimen collection and transportation kit sample) may be placed into an airlock of an anaerobic chamber and introduced into the chamber. With the swab still in place, the tube can be vigorously vortexed for 5 second to release the sample from the swab tip. This is the undiluted or 10⁰ dilution. The swab can then be removed. The sample may undergo a 10-fold serial dilution by transferring 100 microliters into a tube containing 900 microliters of sterile normal saline (0.9%). The sample may be vortex vigorously for 5 seconds and/or mixed by inversion. This is the 10⁻¹ dilution. 100 microliters of the 10⁻¹ dilution may be transferred to a new tube containing 900 microliters of sterile normal saline (0.9%). The sample may be vortex vigorously for 5 seconds and/or mixed by inversion. This is the 10⁻² dilution. The process may be repeated until the specimen has been diluted out to a 10⁻⁶ dilution.

In triplicate, spread plates can be prepared on CDC Anaerobe 5% sheep blood agar, De Man, Rogosa and Sharpe (MRS) agar, and 50% MRS agar plates from the following dilution tubes: 10⁻³, 10⁻⁴, 10⁻⁵. Briefly, from the appropriate tube, 100 μL may be transferred directly onto the surface of an agar plate and spread the inoculum across the plate surface using a sterile L-shaped spreader and a plate rotator. The plates may be incubated at 35° C. under anaerobic conditions for 24-120 hrs. The plates may be checked daily for emergence of new colonies. If plates begin to dry out, wrap them in parafilm. Colonies may be observed and colony counts and morphology may be recorded (which may include taking a capture photo of plates to assist with documentation of colony morphology). Individual colonies for further characterization may be selected and streaked on new plates. If desired, selective media may be utilized such as Bifidobacterium selective agar (BSA), Enterococcosel agar (ECA), Cetrimide Agar (CA), Mannitol Salt Agar (MSA), or the like.

In some instances, gram staining may be utilized. When doing so, a specimen may be applied to a glass slide. If staining a liquid culture, a sterile inoculating loop may be used to directly apply culture to the slide as a smear. If staining a colony from a plate, a drop of sterile saline may be applied to the center of a slide using an inoculating loop, and then a small amount of a single colony may be collected and added to the drop. If possible, the drop and sample can be gently mixed to yield a thin, uniform smear. The drop may be allowed to dry. The slides can be methanol fixed by flooding with absolute methanol for 1-2 minutes, and then rinsing with tap water. The fixed smear may be flooded with the primary stain (e.g., crystal violet) for 1 minute. The primary stain may be removed by gently rinsing with tap water. The fixed smear may be flooded with a secondary stain/mordant (e.g., gram iodine) for 1 minute, followed by gently rinsing with tap water. The slide can be decolorized until the solvent running from the slide is colorless (e.g., about 3-60 seconds) and the slide can be washed gently with tap water. The slide can then be flooded with counterstain (e.g., safranin; basic fuchsin can be substituted if initial counterstain results are not satisfactory) for 1 minute. The slides can be washed with tap water and allowed to dry. The smear can be exampled under an oil immersion lens and the results/observation may be recorded.

Other sample testing can be performed including catalase testing, which may include the addition of hydrogen peroxide to differentiate between Staphylococci and Streptococci. Another sample test that may be performed may include a coagulase test, which may include the addition of plasma to differentiate between Staphylococci and Streptococci.

Isolates that are determined to be Lactobacillus may be further characterized to determine their antibiotic susceptibility profiles. This may include suspending Lactobacillus colonies from an overnight plate in broth comprised of 90% Iso-sensitest broth and 10% MRS broth until the suspension is equivalent to a McFarland standard of 1. Next, a sterile swab may be dipped in the inoculum and squeezed against the wall of the tube to eliminate excess liquid. The swab may be streaked over the entire agar surface (rotating two or more times to ensure even distribution). After allowing excess moisture to be absorbed, a minimum inhibitory concentration (MIC) test strip can be applied (e.g., which may include applying the strip with the scale facing upwards and the code of the strip to the outside of the place). The strip may be pressed onto the surface of the agar while ensuring that the whole length of the antibiotic gradient is in complete contact with the agar surface, repositioning if necessary). The plate can be inverted and incubated at 35° C. for 24-48 hours (or longer). The results can be interpreted.

Isolated strains can be cryopreserved and stored. This may include inoculating an overnight culture with a well-isolated colony selected from a streak plate. If many colonies are being selected, a 96-well plate format may be used. A suitable broth may be used (e.g., Lactobacillus may likely use MRS). The culture may be allowed to incubate at 35° C. overnight in an anaerobic chamber incubator. If the culture is not turbid by the following morning, additional incubation time may be utilized. Once culture appears visually turbid, 200-300 microliters may be transferred to a new 96-well plate containing a cryoprotectant (e.g., 50-60 microliters of 50% glycerol). The final concentration of glycerol may be 10%. The 96-well plates may be sealed and stored in a −80° C. freezer.

As suggested herein, individual microbe strains (e.g., individual strains or species of bacteria) may be isolated from the swabs. This may include one or more processes such as plating onto agar media selective for the desired strains and subsequently “purifying” strains as single-colony isolates. The individual microbe strains may be grown/cultured and be species-identified/characterized by sequencing, biochemical tests, colony morphology, cellular morphology, microscopic mortality, and susceptibility to different representative antibiotics. The microbe strains may vary. In some instances, the microbe strains may include one or more of Lactobacillus crispatus, Lactobacillus iners, Lactobacillus gasseri, Lactobacillus jensenii, Lactobacillus reuteri, Lactobacillus acidophilus, Lactobacillus vaginalis, Lactobacillus rhamnosus, Lactobacillus johnsonii, Lactobacillus helveticus, Lactobacillus plantarum, Lactobacillus fermentum, Lactobacillus salivarius, Lactobacillus delbrueckii, Gardnerella vaginalis, non-Lactobacillus bacteria, Atopobium vaginae, Prevotella bivia, other species of these genera, Megasphaera, Sneathia, Dialister, Peptoniphilus genera, fungi, combinations thereof, and the like.

Vaginal microbiota compositions are contemplated that include a single isolated strain or mixtures of two or more isolated strain. An example composition may include about 20-85% Lactobacillus crispatus, or about 25-75% Lactobacillus crispatus, or about 30-60% Lactobacillus crispatus, or about 40% Lactobacillus crispatus. In some of these and in other instances (e.g., in addition to any one or more of the microorganisms listed above or in the alternative), an example composition may include about 10-50% Lactobacillus iners, or about 10-40% Lactobacillus iners, or about 15-30% Lactobacillus iners, or about 20% Lactobacillus iners. In some of these and in other instances (e.g., in addition to any one or more of the microorganisms listed above or in the alternative), an example composition may include about 1-10% Lactobacillus gasseri, or about 2-8% Lactobacillus gasseri, or about 5% Lactobacillus gasseri. In some of these and in other instances (e.g., in addition to any one or more of the microorganisms listed above or in the alternative), an example composition may include about 1-10% Lactobacillus jensenii, or about 2-8% Lactobacillus jensenii, or about 5% Lactobacillus jensenii. In some of these and in other instances (e.g., in addition to any one or more of the microorganisms listed above or in the alternative), an example composition may include about 0.1-5% Lactobacillus reuteri, or about 0.5-4% Lactobacillus reuteri, or about 1% Lactobacillus reuteri. In some of these and in other instances (e.g., in addition to any one or more of the microorganisms listed above or in the alternative), an example composition may include about 0.1-5% Lactobacillus acidophilus, or about 0.5-4% Lactobacillus acidophilus, or about 1% Lactobacillus acidophilus. In some of these and in other instances (e.g., in addition to any one or more of the microorganisms listed above or in the alternative), an example composition may include about 0.1-5% Lactobacillus vaginalis, or about 0.5-4% Lactobacillus vaginalis, or about 1% Lactobacillus vaginalis. In some of these and in other instances (e.g., in addition to any one or more of the microorganisms listed above or in the alternative), an example composition may include about 0.1-5% Lactobacillus rhamnosus, or about 0.5-4% Lactobacillus rhamnosus, or about 1% Lactobacillus rhamnosus. In some of these and in other instances (e.g., in addition to any one or more of the microorganisms listed above or in the alternative), an example composition may include about 0.1-5% Lactobacillus johnsonii, or about 0.5-4% Lactobacillus johnsonii, or about 1% Lactobacillus johnsonii. In some of these and in other instances (e.g., in addition to any one or more of the microorganisms listed above or in the alternative), an example composition may include about 0.1-5% Lactobacillus helveticus, or about 0.5-4% Lactobacillus helveticus, or about 1% Lactobacillus helveticus. In some of these and in other instances (e.g., in addition to any one or more of the microorganisms listed above or in the alternative), an example composition may include about 0.1-5% Lactobacillus plantarum, or about 0.5-4% Lactobacillus plantarum, or about 1% Lactobacillus plantarum. In some of these and in other instances (e.g., in addition to any one or more of the microorganisms listed above or in the alternative), an example composition may include about 0.1-5% Lactobacillus fermentum, or about 0.5-4% Lactobacillus fermentum, or about 1% Lactobacillus fermentum. In some of these and in other instances (e.g., in addition to any one or more of the microorganisms listed above or in the alternative), an example composition may include about 0.1-5% Lactobacillus salivarius, or about 0.5-4% Lactobacillus salivarius, or about 1% Lactobacillus salivarius. In some of these and in other instances (e.g., in addition to any one or more of the microorganisms listed above or in the alternative), an example composition may include about 0.1-5% Lactobacillus delbrueckii, or about 0.5-4% Lactobacillus delbrueckii, or about 1% Lactobacillus delbrueckii.

An example composition may include about 40% Lactobacillus crispatus, about 20% Lactobacillus iners, about 5% Lactobacillus gasseri, about 5% Lactobacillus jensenii, about 1% Lactobacillus reuteri, about 1% Lactobacillus acidophilus, about 1% Lactobacillus vaginalis, about 1% Lactobacillus rhamnosus, about 1% Lactobacillus johnsonii, about 1% Lactobacillus helveticus, about 1% Lactobacillus plantarum, about 1% Lactobacillus fermentum, about 1% Lactobacillus salivarius, and about 1% Lactobacillus delbrueckii.

An example composition may include Lactobacillus crispatus, Lactobacillus gasseri, Lactobacillus jensenii, and Lactobacillus rhamnosus. For example, the composition may include about 20-85% Lactobacillus crispatus, or about 25-75% Lactobacillus crispatus, or about 30-60% Lactobacillus crispatus, or about 40% Lactobacillus crispatus. The composition may include about 1-10% Lactobacillus gasseri, or about 2-8% Lactobacillus gasseri, or about 5% Lactobacillus gasseri. The composition may include about 1-10% Lactobacillus jensenii, or about 2-8% Lactobacillus jensenii, or about 5% Lactobacillus jensenii. The composition may include about 0.1-5% Lactobacillus rhamnosus, or about 0.5-4% Lactobacillus rhamnosus, or about 1% Lactobacillus rhamnosus. In some instances, each of the components may be lyophilized. The total number of microorganisms in the example composition may be on the order of about 1×10⁵ to 1×10¹⁵ CFU, or about 1×10⁶ to 1×10¹² CFU, or about 1×10⁷ to 1×10¹⁰ CFU. The composition may be disposed in a suitable delivery vehicle such as a capsule, suppository, soluble shell, and/or the like. In some instances, the composition may be administered orally. In other instances, the composition may be administered locally (e.g., by inserting the capsule, suppository, soluble shell, etc. into the vagina).

The example compositions may include strains of Lactobacillus crispatus, Lactobacillus gasseri, Lactobacillus jensenii, and Lactobacillus rhamnosus with desirable characteristics. For example, strains of each of the components may be selected to have a pH reducing activity, produce lactic acid, have an inhibitory activity on other microorganisms, having other unique activities, combinations thereof, and/or the like.

An example composition may include a mixture of lyophilized Lactobacillus crispatus, lyophilized Lactobacillus gasseri, lyophilized Lactobacillus jensenii, and lyophilized Lactobacillus rhamnosus. Each of the components may be isolated from a donor. For example, the composition may include about 20-85% Lactobacillus crispatus, or about 25-75% Lactobacillus crispatus, or about 30-60% Lactobacillus crispatus, or about 40% Lactobacillus crispatus. The composition may include about 1-10% Lactobacillus gasseri, or about 2-8% Lactobacillus gasseri, or about 5% Lactobacillus gasseri. The composition may include about 1-10% Lactobacillus jensenii, or about 2-8% Lactobacillus jensenii, or about 5% Lactobacillus jensenii. The composition may include about 0.1-5% Lactobacillus rhamnosus, or about 0.5-4% Lactobacillus rhamnosus, or about 1% Lactobacillus rhamnosus. Each of the components may be derived/isolated from a donor specimen. The total number of microorganisms in the example composition may be on the order of about 1×10⁵ to 1×10¹⁵ CFU, or about 1×10⁶ to 1×10¹² CFU, or about 1×10⁷ to 1×10¹⁰ CFU. The composition may be disposed in a suitable delivery vehicle such as a capsule, suppository, soluble shell, and/or the like. In some instances, the composition may be administered orally. In other instances, the composition may be administered locally (e.g., by inserting the capsule, suppository, soluble shell, etc. into the vagina).

An example composition may include a mixture of lyophilized Lactobacillus crispatus, lyophilized Lactobacillus gasseri, lyophilized Lactobacillus jensenii, and lyophilized Lactobacillus rhamnosus. Each of the components may be isolated from a donor. For example, the composition may include about 20-85% Lactobacillus crispatus, or about 25-75% Lactobacillus crispatus, or about 30-60% Lactobacillus crispatus, or about 40% Lactobacillus crispatus. The composition may include about 1-10% Lactobacillus gasseri, or about 2-8% Lactobacillus gasseri, or about 5% Lactobacillus gasseri. The composition may include about 1-10% Lactobacillus jensenii, or about 2-8% Lactobacillus jensenii, or about 5% Lactobacillus jensenii. The composition may include about 0.1-5% Lactobacillus rhamnosus, or about 0.5-4% Lactobacillus rhamnosus, or about 1% Lactobacillus rhamnosus. Each of the components may be derived from a bacterial culture. The total number of microorganisms in the example composition may be on the order of about 1×10⁵ to 1×10¹⁵ CFU, or about 1×10⁶ to 1×10¹² CFU, or about 1×10⁷ to 1×10¹⁰ CFU. The composition may be disposed in a suitable delivery vehicle such as a capsule, suppository, soluble shell, and/or the like. In some instances, the composition may be administered orally. In other instances, the composition may be administered locally (e.g., by inserting the capsule, suppository, soluble shell, etc. into the vagina).

An example composition may include a mixture of lyophilized Lactobacillus crispatus, lyophilized Lactobacillus gasseri, lyophilized Lactobacillus jensenii, and lyophilized Lactobacillus rhamnosus. Each of the components may be isolated from a donor. For example, the composition may include about 20-85% Lactobacillus crispatus, or about 25-75% Lactobacillus crispatus, or about 30-60% Lactobacillus crispatus, or about 40% Lactobacillus crispatus. The composition may include about 1-10% Lactobacillus gasseri, or about 2-8% Lactobacillus gasseri, or about 5% Lactobacillus gasseri. The composition may include about 1-10% Lactobacillus jensenii, or about 2-8% Lactobacillus jensenii, or about 5% Lactobacillus jensenii. The composition may include about 0.1-5% Lactobacillus rhamnosus, or about 0.5-4% Lactobacillus rhamnosus, or about 1% Lactobacillus rhamnosus. One or more of the components may be derived/isolated from a donor specimen and one or more the components may be derived from a bacterial culture. The total number of microorganisms in the example composition may be on the order of about 1×10⁵ to 1×10¹⁵ CFU, or about 1×10⁶ to 1×10¹² CFU, or about 1×10⁷ to 1×10¹⁰ CFU. The composition may be disposed in a suitable delivery vehicle such as a capsule, suppository, soluble shell, and/or the like. In some instances, the composition may be administered orally. In other instances, the composition may be administered locally (e.g., by inserting the capsule, suppository, soluble shell, etc. into the vagina).

The example compositions may include strains of Lactobacillus crispatus, Lactobacillus gasseri, Lactobacillus jensenii, and Lactobacillus rhamnosus with desirable characteristics. For example, strains of each of the components may be selected to have a pH reducing activity, produce lactic acid, have an inhibitory activity on other microorganisms, having other unique activities, combinations thereof, and/or the like.

As indicated above, the total number of microorganisms in the example composition may be on the order of about 1×10⁵ to 1×10¹⁵ CFU, or about 1×10⁶ to 1×10¹² CFU, or about 1×10⁷ to 1×10¹⁰ CFU. This may correspond to the total number of microorganisms in a single capsule, suppository, soluble shell, and/or the like. Alternatively, this may correspond to the total number of microorganisms in a suitable dosage of the composition (e.g., which may include one or more capsules, suppositories, soluble shells, and/or the like).

A single capsule, suppository, soluble shell, and/or the like may be described as an encapsulated drug product or dose, which may be administered to a patient. This may include administering the dose to a patient using a suitable dosing regimen. This may include administering one or more doses to the patient. For example, the 1, 2, 3, 4, 5, 6, 7, 8, 9, 10, 11, 12, 13, 14, 15, 16, 17, 18, 19, 20, or more doses may be administered to the patient. In some of these and in other instances, the encapsulated drug products may be administered to the patient on one or more days. For example, the doses may be administered over 1, 2, 3, 4, 5, 6, 7, 8, 9, 10, 11, 12, 13, 14, or more days. In some of these and in other instances, the doses may be administered to the patient one or more times per day to the patient. For example, the doses may be administered 1, 2, 3, 4, 5, 6, or more times per day to the patient.

One example dosing regimen may include administering two doses to the patient two times per day for two days. Another example dosing regimen may include administering four doses to the patient two times per day for two days. Another example dosing regimen may include administering four doses to the patient two times per day for four days. Another example dosing regimen may include two doses per day to the patient for four days. Another example dosing regimen may include one dose per day to the patient for eight days. These are just examples. In at least some of these examples, each dose may include a lyophilized material comprising a mixture of Lactobacillus crispatus, Lactobacillus gasseri, Lactobacillus jensenii, and Lactobacillus rhamnosus.

In at least some instances, the vaginal microbiota composition may be free of bacteria from the genus Sneathia, Megasphaera, Atopobium, Peptoniphilus, Dialister, Prevotella, and/or Gardnerella. For example, the vaginal microbiota composition may be free of bacteria from the genera Atopobium, Gardnerella, and Prevotella.

The vaginal microbiota compositions disclosed herein may be used to treat a number of different health conditions. For example, the vaginal microbiota compositions may be used for treating bacterial vaginosis, treating candidiasis infections, treating human papilloma virus infections, treating urinary tract infections, treating sexually transmitted infections, treating gynecological cancers (e.g., cervical cancer), reducing and/or preventing preterm birth, reducing and/or preventing miscarriages, treating infertility, treating interstitial cystitis, treating polycystic ovary syndrome, and the like.

Vaginal microbiota compositions are also contemplated that include the combination of a full spectrum vaginal microbiota combined or mixed with one or more additional microbes (e.g., such as those collected and isolated from donors). For the purposes of this disclosure, the full spectrum vaginal microbiota may be understood to be a collection of microorganisms present in the vagina of a typical female. The full spectrum vaginal microbiota is not purposefully manipulated to change the presence or absence of any particular microorganism in the sample and, instead, is intended to represent the full population of organisms in the sample. It can be appreciated that the makeup of the vaginal microbiota may differ form person to person. Because of this, the full spectrum vaginal microbiota may have differences. In some instances, vaginal microbiota compositions are contemplated that include full spectrum vaginal microbiota that is combined, mixed, or doped with one or more of Lactobacillus crispatus, Lactobacillus iners, Lactobacillus gasseri, Lactobacillus jensenii, Lactobacillus reuteri, Lactobacillus acidophilus, Lactobacillus vaginalis, Lactobacillus rhamnosus, Lactobacillus johnsonii, Lactobacillus helveticus, Lactobacillus plantarum, Lactobacillus fermentum, Lactobacillus salivarius, Lactobacillus delbrueckii. In at least some instances, both the full spectrum vaginal microbiota and the additional microbes are sourced from vaginal microbiota donors.

The bacteria/microbes may be disposed within a suitable vessel such as a capsule, suppository, soluble shell, and/or the like for oral or local delivery. In some instances, a simulated vaginal fluid may be incorporated into the vaginal microbiota composition. In some of these and in other instances, the vaginal microbiota composition may include glycogen. The total volume contained within the vessel/suppository may be on the order of about 100 microliters to about 10 milliliters.

In at least some instances, the bacteria/microbes in the vaginal microbiota composition may include lyophilized bacteria/microbes. This may include a lyophilization process in which bacteria/microbes are freeze-dried. The bacteria/microbes and/or the lyophilized bacteria/microbes may be disposed within a suitable vessel such as a capsule, suppository, soluble shell, and/or the like for oral or local delivery.

Treatment of patients may include the administration of a vaginal microbiota composition to the patient. One aim of the treatment may be to normalize the vaginal microbiota of the patient by effectively replacing the vaginal microbiota of the patient with the vaginal microbiota composition. This may include administering the vaginal microbiota composition directly to the vagina of the patient by implantation, suppository, or another suitable route of administration.

In at least some instances, a plurality of vaginal microbiota compositions may be stored or banked in a suitable storage device/facility. The storage device may include temperature control device such as a refrigeration device (e.g., a 4° C. refrigeration device), freezer (e.g. a −20° C. freezer), deep freezer (e.g., a −80° C. freezer), or the like. In some instances, an indexing system may also be coupled to or otherwise associated with the vaginal microbiota composition bank. The indexing system may include data such as donor data that can be used to match a vaginal microbiota composition from a given donor with a suitable patient. The donor data may include age at collection, reproductive status (non-menopausal, menopausal) at collection, race, menstrual stage at collection, history of bacterial vaginosis, history of candidiasis infection, history of human papilloma virus, other medical history (e.g., history of C. difficile infection, history of cancer diagnosis/treatment, etc.), combinations thereof, and/or the like. A clinician may utilize donor data to tailor a treatment regime to a particular patient. For example, a patient with bacterial vaginosis may be treated with a vaginal microbiota composition derived from a donor with a history of successful amelioration of bacterial vaginosis.

In some instances, donor data may include pregnancy history. For example, some donors may have a history of a high probability of becoming pregnant when seeking pregnancy or otherwise have a real or perceived elevated fertility. Vaginal microbiota compositions including microbes from such donors may be administered to patients experiencing fertility challenges in order to attempt to increase or boost the fertility of the patient. In such cases, the vaginal microbiota composition may be administered as an oral capsule. Alternatively, the vaginal microbiota composition may be administered as a transplant and/or otherwise inserted directly into the vagina of the patient.

U.S. Pat. No. 9,675,648 is herein incorporated by reference.

U.S. Pat. No. 9,629,881 is herein incorporated by reference.

U.S. Pat. No. 10,226,431 is herein incorporated by reference.

U.S. Patent Application Pub. No. US 2018/0289750 is herein incorporated by reference.

### EXAMPLES

The disclosure may be further clarified by reference to the following Examples, which are prophetic in nature and serve to exemplify some embodiments, and not to limit the disclosure in any way.

### Example 1

A vaginal microbiota composition can be manufactured. The vaginal microbiota composition includes a mixture of bacterial that includes about 40% Lactobacillus crispatus, about 20% Lactobacillus iners, about 5% Lactobacillus gasseri, about 5% Lactobacillus jensenii, about 1% Lactobacillus reuteri, about 1% Lactobacillus acidophilus, about 1% Lactobacillus vaginalis, about 1% Lactobacillus rhamnosus, about 1% Lactobacillus johnsonii, about 1% Lactobacillus helveticus, about 1% Lactobacillus plantarum, about 1% Lactobacillus fermentum, about 1% Lactobacillus salivarius, and about 1% Lactobacillus delbrueckii.

The mixture of bacteria can be disposed in a suppository. The total volume (e.g., the volume of material including the mixture of bacteria) contained within the suppository may be on the order of about 100 microliters to about 10 milliliters.

The suppository can be administered to a patient. For example, the suppository can be administered to a patient to treat one or more of bacterial vaginosis, a candidiasis infection, a human papilloma virus infection, a urinary tract infection, a sexually transmitted infection, and a gynecological cancer.

The suppository can be administered to a patient. For example, the suppository can be administered to a patient to prevent preterm birth, prevent miscarriages, treat infertility, treat interstitial cystitis, treating polycystic ovary syndrome, and/or combinations thereof.

### Example 2—Example Lactobacillus jensenii Strain for Use in a Vaginal Microbiota Composition

A strain of Lactobacillus jensenii, designated strain 8001-D01-M006, was isolated from a donor's specimen deposited during a donation (e.g., donated as described herein). The donor was qualified by donating a vaginal swab a urine specimen, which were submitted to Fairview Diagnostics Laboratory for Gram staining and Nugent scoring (GRAM) and testing for the following STIs: Trichomonas vaginalis (TVPCR), Neisseria gonorrhoeae (GCPCR), and Chlamydia trachomatis (CHPCR).

A set culture cell density was not targeted during the preparation of a working cell stock for 8001-D01-M006, but rather an overnight culture was started by selecting a single well isolated colony from a previously prepared streak plate. This overnight culture was diluted 1:5 into fresh broth the next morning and allowed to grow for at least 4 hours, after which the culture was combined with glycerol to prepare 10% glycerol stocks. In general, preparation of glycerol stocks in this manner has resulted in stocks with a cell density of about 1×10⁹ CFU/ml.

OmniGene and ESwab Vaginal swab Specimens (8001-D01) were provided for metagenomics sequencing (OmniGene), and culturing and qPCR identity testing (ESwab). ESwab specimen 8001-D01 for culturing and qPCR identity testing was introduced into the anaerobic chamber. ESwab specimen was vortexed briefly before removing 50 μL of sample. Sample aliquot was pelleted by centrifugation. DNA was extracted from the pellet, and sample was evaluated by qPCR to determine Lactobacillus species identity.

An additional 100 μL of sample was removed from the original ESwab specimen. The aliquot was 10-fold serially diluted, and then 100 μL from the 10⁻³, 10⁻⁴, and 10⁻⁵ dilutions were spread plated on MRS agar and then incubated at 35° C. for 24-72 hours.

Presumptive Lactobacilli were identified on the agar plates by colony morphology and gram staining. Individual colonies with colony morphology matching the presumptive Lactobacillus colony morphology were picked using a sterile toothpick and then patched onto fresh MRS agar, LAMVAB agar (media selective for Lactobacillus), before inoculating MRS broth in the well of a 96-deep well plate. The 96-deep well plate containing lactobacilli grown in MRS broth was incubated for about 24 hours. The patch plates were incubated at 35° C. for 24-72 hours.

Strain 8001-D01-M006 was picked from one of the original MRS agar dilution plates, spread on grid spot #6 on the MRS agar patch plate, grid spot #6 on the LAMVAB plate, and inoculated into the MRS broth in well F7. The broth culture for strain 8001-D01-M006 growing in well F7 of the 96-deep well plate was transferred to well F7 of a 96-well PCR plate, and well F7 of two 96-well microtiter plates (containing 50% glycerol in the well so that upon addition of the 8001-D01-M006 culture, the final glycerol concentration was 10%). The culture aliquoted into the 96-well PCR plate was pelleted, DNA was extracted, and samples were tested by qPCR to determined Lactobacillus species identity (when possible). Strain 8001-D01-M006 was determined to be Lactobacillus jensenii by qPCR. The 2×96-well microtiter plates containing culture and 10% glycerol (cryopreservation plates) were sealed with an adhesive foil seal and then stored frozen at or below −70° C.

Strain 8001-D01-M006 was retrieved from frozen 96-well microtiter plate for expansion and characterization. One 96-well microtiter plate containing culture and 10% glycerol was introduced into the anaerobic chamber and allowed to thaw at room temperature for 1 hour. The culture in well F7 was gently mixed by pipetting and then was streaked for isolation on MRS agar. Plate was incubated at 35° C. for 24-72 hours.

A single well-isolated colony was selected from the streak plate and used to inoculate an overnight culture in pre-reduced MRS broth (18-24 hours at 35° C.). The next day, the overnight culture was diluted into fresh MRS broth, allowed to incubate for about 4 hours before being combined with 50% glycerol (final concentration of 10% glycerol, aliquoted into cryovials (1 mL per vial) and stored frozen at or below −70° C.

Strain 8001-D01-M006 was isolated and propagated using pre-reduced MRS agar and broth. The lots of media used are recorded on the P249-01 Specimen Culturing Worksheet for specimen 8001-D01. The completed media preparation worksheets for each lot of MRS broth and MRS agar are stored in the P249 binder. Briefly, the MRS agar and MRS broth growth media were prepared in-house using Hardy Diagnostic's Criterion™ Lactobacilli MRS broth which contains the following components (gram weight per liter): Dextrose (20.0 g), Peptic Digest of Animal Tissue (10.0 g), Beef Extract (10.0 g), Yeast Extract (5.0 g), Sodium Acetate (5.0 g), Disodium Phosphate (2.0 g), Ammonium Citrate (2.0 g), Tween 80 (1.0 g), Magnesium Sulfate (0.1 g), and Manganese Sulfate (0.05 g). MRS agar plates use the same Criterion™ Lactobacilli MRS broth as a base and include 15 g/L of BD Difco agar. MRS broth and agar also contain 0.5 g/L of L-cysteine.

This strain 8001-D01-M006 has been identified by qPCR and Strainview sequencing. The qPCR method detects the Lactobacillus genus and seven (7) different Lactobacillus species (L. crispatus, L. gasseri, L. jensenii, L. iners, L. acidophilus, L. delbrueckii, and L. helveticus) by comparing melting curve profiles for amplicons generated from unknown isolates and known positive control strains. Strainview sequencing was performed by Diversigen. The strain was found to contain zero antibiotic resistance genes and zero virulence genes when analyzed using Strainview pipelines.

Strain 8001-D01-M006 may be an example of a suitable Lactobacillus jenseniis strain that can be utilized for a vaginal microbiota composition.

### Example 3—Example Lactobacillus gasseri Strain for Use in a Vaginal Microbiota Composition

A strain of Lactobacillus gasseri, designated strain 8018-IN-M001, was isolated from a donor's specimen deposited during a donation (e.g., donated as described herein). The donor has been qualified by donating a vaginal swab a urine specimen, which were submitted to Fairview Diagnostics Laboratory for Gram staining and Nugent scoring (GRAM) and testing for the following STIs: Trichomonas vaginalis (TVPCR), Neisseria gonorrhoeae (GCPCR), and Chlamydia trachomatis (CHPCR).

A set culture cell density was not targeted during the preparation of a working cell stock for 8018-IN-M001, but rather an overnight culture was started by selecting a single well isolated colony from a previously prepared streak plate. This overnight culture was diluted 1:5 into fresh broth the next morning and allowed to grow for at least 4 hours, after which the culture was combined with glycerol to prepare 10% glycerol stocks. In general, preparation of glycerol stocks in this manner has resulted in stocks with a cell density of about 1×10⁸ to 7×10⁸ CFU/ml.

OmniGene and ESwab Vaginal swab Specimens (8018-IN) were provided for metagenomics sequencing (OmniGene), and culturing and qPCR identity testing (ESwab). ESwab specimen 8018-IN for culturing and qPCR identity testing was introduced into the anaerobic chamber. ESwab specimen was vortexed briefly before removing 50 μL of sample. Sample aliquot was pelleted by centrifugation. DNA was extracted from the pellet, and sample was evaluated by qPCR to determine Lactobacillus species identity.

An additional 100 μL of sample was removed from the original ESwab specimen. The aliquot was 10-fold serially diluted, and then 100 μL from the 10⁻³, 10⁻⁴, and 10⁻⁵ dilutions were spread plated on MRS agar and then incubated at 35° C. for 24-72 hours.

Presumptive Lactobacilli were identified on the agar plates by colony morphology and gram staining. Individual colonies with colony morphology matching the presumptive Lactobacillus colony morphology were picked using a sterile toothpick and then patched onto fresh MRS agar, LAMVAB agar (media selective for Lactobacillus), before inoculating MRS broth in the well of a 96-deep well plate. The 96-deep well plate containing lactobacilli grown in MRS broth was incubated for about 24 hours. The patch plates were incubated at 35° C. for 24-72 hours.

Strain 8018-IN-M001 was picked from one of the original MRS agar dilution plates, spread on grid spot #1 on the MRS agar patch plate, grid spot #1 on the LAMVAB plate, and inoculated into the MRS broth in well A7. The broth culture for strain 8018-IN-M001 growing in well A7 of the 96-deep well plate was transferred to well A7 of a 96-well PCR plate, and well A7 of two 96-well microtiter plates (containing 50% glycerol in the well so that upon addition of the 8018-IN-M001 culture, the final glycerol concentration was 10%). The culture aliquoted into the 96-well PCR plate was pelleted, DNA was extracted, and samples were tested by qPCR to determined Lactobacillus species identity (when possible). Strain 8018-IN-M001 was determined to be Lactobacillus gasseri by qPCR. The 2×96-well microtiter plates containing culture and 10% glycerol (cryopreservation plates) were sealed with an adhesive foil seal and then stored frozen at or below −70° C.

Strain 8018-IN-M001 was retrieved from frozen 96-well microtiter plate for expansion and characterization. One 96-well microtiter plate containing culture and 10% glycerol was introduced into the anaerobic chamber and allowed to thaw at room temperature for 1 hour. The culture in well A7 was gently mixed by pipetting and then was streaked for isolation on MRS agar. Plate was incubated at 35° C. for 24-72 hours.

A single well-isolated colony was selected from the streak plate and used to inoculate an overnight culture in pre-reduced MRS broth (18-24 hours at 35° C.). The next day, the overnight culture was diluted into fresh MRS broth, allowed to incubate for about 4 hours before being combined with 50% glycerol (final concentration of 10% glycerol, aliquoted into cryovials (1 mL per vial) and stored frozen at or below −70° C.

Strain 8018-IN-M001 was isolated and propagated using pre-reduced MRS agar and broth. The lots of media used are recorded on the P249-01 Specimen Culturing Worksheet for specimen 8018-IN. The completed media preparation worksheets for each lot of MRS broth and MRS agar are stored in the P249 binder. Briefly, the MRS agar and MRS broth growth media were prepared in-house using Hardy Diagnostic's Criterion™ Lactobacilli MRS broth which contains the following components (gram weight per liter): Dextrose (20.0 g), Peptic Digest of Animal Tissue (10.0 g), Beef Extract (10.0 g), Yeast Extract (5.0 g), Sodium Acetate (5.0 g), Disodium Phosphate (2.0 g), Ammonium Citrate (2.0 g), Tween 80 (1.0 g), Magnesium Sulfate (0.1 g), and Manganese Sulfate (0.05 g). MRS agar plates use the same Criterion™ Lactobacilli MRS broth as a base and include 15 g/L of BD Difco agar. MRS broth and agar also contain 0.5 g/L of L-cysteine.

This strain 8018-IN-M001 has been identified by qPCR and Strainview sequencing. The qPCR method detects the Lactobacillus genus and seven (7) different Lactobacillus species (L. crispatus, L. gasseri, L. jensenii, L. iners, L. acidophilus, L. delbrueckii, and L. helveticus) by comparing melting curve profiles for amplicons generated from unknown isolates and known positive control strains. Strainview sequencing was performed by Diversigen. The strain was found to contain zero antibiotic resistance genes and zero virulence genes when analyzed using Strainview pipelines.

Strain 8018-IN-M001 may be an example of a suitable Lactobacillus gasseri strain that can be utilized for a vaginal microbiota composition.

### Example 4—Example Lactobacillus crispatus Strain for Use in a Vaginal Microbiota Composition

A strain of Lactobacillus crispatus, designated strain 8001-D01-M004, was isolated from a donor's specimen deposited during a donation (e.g., donated as described herein). The donor has been qualified by donating a vaginal swab a urine specimen, which were submitted to Fairview Diagnostics Laboratory for Gram staining and Nugent scoring (GRAM) and testing for the following STIs: Trichomonas vaginalis (TVPCR), Neisseria gonorrhoeae (GCPCR), and Chlamydia trachomatis (CHPCR).

A set culture cell density was not targeted during the preparation of a working cell stock for 8001-D01-M004, but rather an overnight culture was started by selecting a single well isolated colony from a previously prepared streak plate. This overnight culture was diluted 1:5 into fresh broth the next morning and allowed to grow for at least 4 hours, after which the culture was combined with glycerol to prepare 10% glycerol stocks. In general, preparation of glycerol stocks in this manner has resulted in stocks with a cell density of about 1×10⁷ to 1×10⁹ CFU/ml.

OmniGene and ESwab Vaginal swab Specimens (8001-D01) were provided for metagenomics sequencing (OmniGene), and culturing and qPCR identity testing (ESwab). ESwab specimen 8001-D01 for culturing and qPCR identity testing was introduced into the anaerobic chamber. ESwab specimen was vortexed briefly before removing 50 μL of sample. Sample aliquot was pelleted by centrifugation. DNA was extracted from the pellet, and sample was evaluated by qPCR to determine Lactobacillus species identity.

An additional 100 μL of sample was removed from the original ESwab specimen. The aliquot was 10-fold serially diluted, and then 100 μL from the 10⁻³, 10⁻⁴, and 10⁻⁵ dilutions were spread plated on MRS agar and then incubated at 35° C. for 24-72 hours.

Presumptive Lactobacilli were identified on the agar plates by colony morphology and gram staining. Individual colonies with colony morphology matching the presumptive Lactobacillus colony morphology were picked using a sterile toothpick and then patched onto fresh MRS agar, LAMVAB agar (media selective for Lactobacillus), before inoculating MRS broth in the well of a 96-deep well plate. The 96-deep well plate containing lactobacilli grown in MRS broth was incubated for about 24 hours. The patch plates were incubated at 35° C. for 24-72 hours.

Strain 8001-D01-M004 was picked from one of the original MRS agar dilution plates, spread on grid spot #4 on the MRS agar patch plate, grid spot #4 on the LAMVAB plate, and inoculated into the MRS broth in well D7. The broth culture for strain 8001-D01-M004 growing in well D7 of the 96-deep well plate was transferred to well D7 of a 96-well PCR plate, and well D7 of two 96-well microtiter plates (containing 50% glycerol in the well so that upon addition of the 8001-D01-M004 culture, the final glycerol concentration was 10%). The culture aliquoted into the 96-well PCR plate was pelleted, DNA was extracted, and samples were tested by qPCR to determined Lactobacillus species identity (when possible). Strain 8001-D01-M004 was determined to be Lactobacillus crispatus by qPCR. The 2×96-well microtiter plates containing culture and 10% glycerol (cryopreservation plates) were sealed with an adhesive foil seal and then stored frozen at or below −70° C.

Strain 8001-D01-M004 was retrieved from frozen 96-well microtiter plate for expansion and characterization. One 96-well microtiter plate containing culture and 10% glycerol was introduced into the anaerobic chamber and allowed to thaw at room temperature for 1 hour. The culture in well D7 was gently mixed by pipetting and then was streaked for isolation on MRS agar. Plate was incubated at 35° C. for 24-72 hours.

A single well-isolated colony was selected from the streak plate and used to inoculate an overnight culture in pre-reduced MRS broth (18-24 hours at 35° C.). The next day, the overnight culture was diluted into fresh MRS broth, allowed to incubate for about 4 hours before being combined with 50% glycerol (final concentration of 10% glycerol, aliquoted into cryovials (1 mL per vial) and stored frozen at or below −70° C.

Strain 8001-D01-M004 was isolated and propagated using pre-reduced MRS agar and broth. The lots of media used are recorded on the P249-01 Specimen Culturing Worksheet for specimen 8001-D01. The completed media preparation worksheets for each lot of MRS broth and MRS agar are stored in the P249 binder. Briefly, the MRS agar and MRS broth growth media were prepared in-house using Hardy Diagnostic's Criterion™ Lactobacilli MRS broth which contains the following components (gram weight per liter): Dextrose (20.0 g), Peptic Digest of Animal Tissue (10.0 g), Beef Extract (10.0 g), Yeast Extract (5.0 g), Sodium Acetate (5.0 g), Disodium Phosphate (2.0 g), Ammonium Citrate (2.0 g), Tween 80 (1.0 g), Magnesium Sulfate (0.1 g), and Manganese Sulfate (0.05 g). MRS agar plates use the same Criterion™ Lactobacilli MRS broth as a base and include 15 g/L of BD Difco agar. MRS broth and agar also contain 0.5 g/L of L-cysteine.

This strain 8001-D01-M004 has been identified by qPCR and Strainview sequencing. The qPCR method detects the Lactobacillus genus and seven (7) different Lactobacillus species (L. crispatus, L. gasseri, L. jensenii, L. iners, L. acidophilus, L. delbrueckii, and L. helveticus) by comparing melting curve profiles for amplicons generated from unknown isolates and known positive control strains. Strainview sequencing was performed by Diversigen. The strain was found to contain zero antibiotic resistance genes and zero virulence genes when analyzed using Strainview pipelines.

Strain 8001-D01-M004 may be an example of a suitable Lactobacillus crispatus strain that can be utilized for a vaginal microbiota composition.

### Example 5—Example Lactobacillus rhamnosus Strain for Use in a Vaginal Microbiota Composition

An example strain of Lactobacillus rhamnosus, designated strain BPL5, is disclosed in U.S. Pat. No. 10,588,926, the entire contents of which are herein incorporated by reference.

Strain BPL5 may be an example of a suitable Lactobacillus rhamnosus strain that can be utilized for a vaginal microbiota composition.

### Example 6—Strain pH-Reducing Capacity and Lactic Acid Production when Grown Anaerobically in MRS Broth for 48 Hours

Strains BPL5 (L. rhamnosus; Example 6), 8001-D01-M006 (L. jensenii; Example 2), 8001-D01-M004 (L. crispatus; Example 4), and 8018-IN-M001 (L. gasseri; Example 3) were evaluated for their in vitro ability to lower the pH of MRS broth over time. To evaluate pH-reduction, each strain was inoculated into MRS broth and grown overnight (under anaerobic conditions). The next day, the overnight culture was adjusted to an OD_{600 nm} of approximately 1.0 (which was equivalent to about 1×10⁸ CFU/mL), and then was diluted further into fresh MRS broth (targeting a starting concentration of ˜1×10⁶ CFU/mL (for 8001-D01-M006, 8001-D01-M004, and 8018-IN-M001). Strain BPL5 was tested separately and at a different starting concentration (about 1×10⁷ CFU/mL). All cultures were incubated anaerobically for 48 hours, with samples collected for pH after 0 and 48 hours of growth. All strains were able to significantly reduce the broth pH by 48 hours with the strain of L. rhamnosus and L. crispatus showing the most significant pH-reduction (Table 1). Both the BPL5 (L. rhamnosus) and 8001-D01-M004 (L. crispatus) strains were able to reduce the broth pH to lower than 4.0 by 48 hours (3.78 and 3.77, respectively). The 8001-D01-M006 (L. jensenii) and 8018-IN-M001 (L. gasseri) strains also reduced broth pH by 48 hours (4.02 and 4.06, respectively), but not to the same extent as the L. rhamnosus and L. crispatus strains.

TABLE 1
pH Reduction by Vaginal Lactobacillus
Strains Grown Anaerobically in MRS Broth

| Strain | 0 Hours | 48 Hours |
|---|---|---|
| BPL5 | 6.18 | 3.78 |
| (L. rhamnosus) | | |
| 8001-D01-M006 | 6.34 | 4.02 |
| (L. jensenii) | | |
| 8001-D01-M004 | 6.33 | 3.77 |
| (L. crispatus) | | |
| 8018-IN-M001 | 6.33 | 4.06 |
| (L. gasseri) | | |

Additional samples were collected at the 48-hour time point and filtered to remove bacteria. These cell-free supernatants were then analyzed to detect levels of D- and L-lactic acid. D- and L-lactic acid were quantitated using the D-/L-Lactic Acid (D-/L-Lactate) (Rapid) Assay Kit manufactured by Megazyme (Catalog #K-DLATE). This kit is designed to quantitate both D-and L-lactic acid using enzymatic reactions using lactate dehydrogenase and glutamate. The amount of NADH produced by the oxidation of lactic acid is stoichiometric to the starting concentration of lactic acid. This kit was used following the manufacturer's recommended procedure. All strains tested produced detectable levels of lactic acid (Table 2). The 8001-D01-M004 (L. crispatus) strain produced the greatest level of D- and L-lactic acid (20.9 g/L) and the greatest amount of D-lactic acid (13.5), while the BPL5 (L. rhamnosus) strain produced the greatest amount of L-lactic acid (17.3 g/L) and very little D-lactic acid (0.8 g/L). Both L. crispatus and L. gasseri strains produced high amounts of both lactic acid isomers, with both producing more D-lactic acid than L-lactic acid. The 8001-D01-M006 strain of L. jensenii predominantly produced D-lactic acid (12.4 g/L) and made very little L-Lactic acid (0.3 g/L).

TABLE 2
Lactic Acid Production by Vaginal Lactobacillus Strains

| | | D-Lactic | L-Lactic | Total Lactic |
|---|---|---|---|---|
| | Time | Acid Conc. | Acid Conc. | Acid Conc. |
| Strain | Point | (g/L) | (g/L) | (g/L) |
| BPL5 | 48 Hr | 0.8 | 17.3 | 18.1 |
| (L. rhamnosus) | | | | |
| 8001-D01-M006 | 48 Hr | 12.4 | 0.3 | 12.7 |
| (L. jensenii) | | | | |
| 8001-D01-M004 | 48 Hr | 13.5 | 7.4 | 20.9 |
| (L. crispatus) | | | | |
| 8018-IN-M001 | 48 Hr | 6.7 | 4.3 | 11 |
| (L. gasseri) | | | | |

These results demonstrate that strains BPL5 (L. rhamnosus; Example 6), 8001-D01-M006 (L. jensenii; Example 2), 8001-D01-M004 (L. crispatus; Example 4), and 8018-IN-M001 (L. gasseri; Example 3) may be suitable materials for use in a vaginal microbiota composition.

### Example 7—Example Vaginal Microbiota Composition

An example vaginal microbiota composition may be formed by combining Lactobacillus crispatus, Lactobacillus gasseri, Lactobacillus jensenii, Lactobacillus. rhamnosus. In at least some instances, the Lactobacillus jensenii may be the strain described in Example 2. In at least some instances, the Lactobacillus gasseri may be the strain described in Example 3. In at least some instances, the Lactobacillus crispatus may be the strain described in Example 4. In at least some instances, the Lactobacillus rhamnosus may be the strain described in Example 5.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

1. A vaginal microbiota composition, comprising:
a mixture of bacteria including Lactobacillus crispatus, Lactobacillus gasseri, Lactobacillus jensenii, and Lactobacillus rhamnosus.

2. The vaginal microbiota composition of claim 1, wherein the mixture of bacteria includes 25-75% Lactobacillus crispatus.

3. The vaginal microbiota composition of claim 1, wherein the mixture of bacteria includes 30-60% Lactobacillus crispatus.

4. The vaginal microbiota composition of claim 1, wherein the mixture of bacteria includes 40% Lactobacillus crispatus.

5. The vaginal microbiota composition of claim 1, wherein the mixture of bacteria includes 1-10% Lactobacillus gasseri.

6. The vaginal microbiota composition of claim 1, wherein the mixture of bacteria includes 2-8% Lactobacillus gasseri.

7. The vaginal microbiota composition of claim 1, wherein the mixture of bacteria includes 5% Lactobacillus gasseri.

8. The vaginal microbiota composition of claim 1, wherein the mixture of bacteria includes 1-10% Lactobacillus jensenii.

9. The vaginal microbiota composition of claim 1, wherein the mixture of bacteria includes 2-8% Lactobacillus jensenii.

10. The vaginal microbiota composition of claim 1, wherein the mixture of bacteria includes 5% Lactobacillus jensenii.

11. The vaginal microbiota composition of claim 1, wherein the mixture of bacteria includes 0.1-5% Lactobacillus acidophilus.

12. The vaginal microbiota composition of claim 1, wherein the mixture of bacteria includes 0.1-5% Lactobacillus rhamnosus.

13. The vaginal microbiota composition of claim 1, wherein the mixture of bacteria includes 0.5-4% Lactobacillus rhamnosus.

14. The vaginal microbiota composition of claim 1, wherein the mixture of bacteria includes 1% Lactobacillus rhamnosus.

15. The vaginal microbiota composition of claim 1, wherein the mixture of bacteria is disposed in a capsule.

16. The vaginal microbiota composition of claim 1, wherein the mixture of bacteria is disposed in a suppository.

17. The vaginal microbiota composition of claim 1, wherein the mixture of bacteria is disposed in a soluble shell.

18. The vaginal microbiota composition of claim 1, wherein the mixture of bacteria includes 1×10⁵ to 1×10¹⁵ colony forming units (CFU).

19. The vaginal microbiota composition of claim 1, wherein the mixture of bacteria includes 1×10⁶ to 1×10¹² CFU.

20. The vaginal microbiota composition of claim 1, wherein the mixture of bacteria includes 1×10⁷ to 1×10¹⁰ CFU.

21. The vaginal microbiota composition of claim 1, wherein the mixture of bacteria includes a mixture of lyophilized bacteria.

22. A method for treating an infection, the method comprising:
administering the vaginal microbiota composition of claim 1 to a patient with an infection, wherein the infection includes one or more of bacterial vaginosis, a candidiasis infection, a human papilloma virus infection, a urinary tract infection, a sexually transmitted infection, and a gynecological cancer.
